(19) United States
(12) Patent Application Publication
(10) Pub. No.: US 2026/0130652 A1
(43) Pub. Date: May 14, 2026

(54) FUSION OF ULTRASOUND IMAGES WITH PRE-PROCEDURE STRUCTURAL 3-D IMAGE DATA FOR ULTRASOUND IMAGE-GUIDED PROCEDURES

(71) Applicant: GE Precision Healthcare LLC, Waukesha, WI (US)

(72) Inventors: Bo Martins, Rodovre (DK); Fredrik Gran, Limhamn (SE)

(73) Assignee: GE Precision Healthcare LLC, Waukesha, WI (US)

(21) Appl. No.: 18/941,580

(22) Filed: Nov. 8, 2024

(51) Int. Cl. A61B 8/08; A61B 8/00; A61B 34/10; A61B 90/00

(52) U.S. Cl. CPC A61B 8/5261; A61B 8/4483; A61B 8/463; A61B 8/464; A61B 8/466; A61B 8/483; A61B 8/523; A61B 34/10; A61B 2034/107; A61B 2090/374

(57) ABSTRACT

An ultrasound imaging system includes a single transducer array having a long axis and configured to transmit and receive in either a first mode to acquire a live ultrasound image of a sagittal plane during a procedure or a second mode to acquire 3-D ultrasound data including a plurality of sagittal planes by rotating the single transducer array about an axis that is parallel to the long axis of the transducer array, a resampler configured to resample the 3-D ultrasound data and generate a set of resampled ultrasound planes that are approximately orthogonal to a surface of tissue of interest, a segmentor configured to segment a first contour of the tissue of interest in the set of resampled ultrasound planes, a registration engine configured to register the first contour and a second contour of the tissue of interest in pre-procedure 3-D MR image data and create a fused 3-D image.

### FIELD

The following generally relates to ultrasound imaging, and, more particularly, to fusion of ultrasound images with pre-procedure structural 3-D image data for ultrasound image-guided procedures.

### BACKGROUND

An ultrasound imaging system includes a transducer array that transmits an ultrasound beam into an examination field of view. As the beam traverses structure (e.g., of a sub-portion of an object or subject) in the field of view, portions of the beam are attenuated, scattered, and/or reflected off the structure, with some of the reflections (echoes) traversing back towards the transducer array. The transducer array receives echoes, which are processed to generate an ultrasound image of the portion of the object or subject. The ultrasound image is visually displayed.

Ultrasound imaging is used in a wide range of medical applications. An example of a medical application is ultrasound-guided biopsy or therapy. Generally, a biopsy is a procedure in which a small sample(s) of tissue of interest (e.g., prostate, lung, breast, etc.) is removed for subsequent examination for abnormalities such as cancer cells. For a biopsy, a needle is inserted through the skin and advanced to the target tissue where the sample(s) is taken. With ultrasound-guided biopsy or therapy, ultrasound is used to assist a clinician with locating and/or navigating an instrument to the tissue of interest.

With one approach, during the procedure, a live ultrasound image is combined with pre-procedure structural (e.g., Magnetic Resonance (MR)) 3-D image data to provide an anatomical frame of reference to track advancement of the instrument. To accomplish this, first, the transducer array is utilized to acquire an image of the tissue of interest in the sagittal plane and an image of the tissue of interest in the transverse plane. Then, contours of the tissue of interest segmented in the two orthogonal planes are co-registered with a contour of the tissue from a segmentation of the 3-D MR image data. The segmentation can include various features including lesions. A live ultrasound image is then superimposed with contours of the features in a corresponding plane in the 3-D MR image during the procedure.

Unfortunately, the two orthogonal ultrasound image planes generally are not able to show a specific point of interest simultaneously without moving the transducer array to a different location where the overview of the tissue is reduced. As a consequence, the co-registration of the contours in the two orthogonal ultrasound image planes with the 3-D MR image data may not be accurate, and a current position of the instrument in a live ultrasound image with respect to the anatomical frame of reference provide by the 3-D MR image data may not accurately represent the actual position of the instrument relative to the tissue of interest.

As such, there is an unresolved need for an improved approach that mitigates the above noted and/or other shortcomings of existing approaches for fusing ultrasound images and structural 3-D image data for ultrasound image-guided procedures.

### SUMMARY

Aspects of the application address the above matters, and others. This summary introduces concepts that are described in more detail in the detailed description. It should not be used to identify essential features of the claimed subject matter, nor to limit the scope of the claimed subject matter.

In one aspect, an ultrasound imaging system includes a single transducer array having a long axis and configured to transmit and receive in either a first mode to acquire a live ultrasound image of a sagittal plane during a procedure or a second mode to acquire 3-D volumetric ultrasound data including a plurality of sagittal planes by rotating the single transducer array about an axis that is parallel to the long axis of the transducer array. The ultrasound imaging system further includes a resampler configured to resample the 3-D volumetric ultrasound data and generate a set of resampled ultrasound planes that are approximately orthogonal to a surface of tissue of interest. The ultrasound imaging system further includes a segmentor configured to segment a first contour of the tissue of interest in the set of resampled ultrasound planes. The ultrasound imaging system further includes a registration engine configured to register the first contour and a second contour of the tissue of interest in pre-procedure three-dimensional (3-D) magnetic resonance (MR) image data and create a fused 3-D image. The ultrasound imaging system further includes a rendering engine configured to superimpose the live ultrasound image with contours of features segmented from the pre-procedure 3-D MR image data.

In another aspect, a computer-implemented method includes acquiring, with a single transducer array having a long axis, either a live ultrasound image of a sagittal plane during a procedure or 3-D volumetric ultrasound data while rotating the single transducer array about an axis that is parallel to the long axis of the transducer array. The computer-implemented method further includes resampling the 3-D volumetric ultrasound data to generate a set of resampled ultrasound planes that are approximately orthogonal to a surface of tissue of interest. The computer-implemented method further includes segmenting a first contour of the tissue of interest in the set of resampled ultrasound planes. The computer-implemented method further includes registering the first contour and a second contour of the tissue of interest in 3-D pre-procedure MR image data to create a fused 3-D image. The computer-implemented method further includes superimposing the live ultrasound image with contours of features segmented from the pre-procedure 3-D MR image data on a display monitor.

In another aspect, a computer readable medium is encoded with computer executable instructions, which, when executed by a processor, cause the processor to acquire, with a single transducer array having a long axis, either a live ultrasound image of a sagittal plane during a procedure or 3-D volumetric ultrasound data while rotating the single transducer array about an axis that is parallel to the long axis of the transducer array, resample the 3-D volumetric ultrasound data to generate a set of resampled ultrasound planes that are approximately orthogonal to a surface of tissue of interest, segment a first contour of the tissue of interest in the set of sagittal ultrasound planes, register the first contour and a second contour of the tissue of interest in 3-D pre-procedure MR data to create a fused image, and superimpose the live ultrasound image with contours of features segmented from the pre-procedure 3-D MR image data on a display monitor.

Those skilled in the art will recognize still other aspects of the present application upon reading and understanding the attached description.

### BRIEF DESCRIPTION OF THE DRAWINGS

The application is illustrated by way of example and not limited by the figures of the accompanying drawings in which like references indicate similar elements.

FIG. 1 schematically illustrates a non-limiting example of an ultrasound imaging system that includes a fusion module, in accordance with an aspect of an embodiment(s) herein.

FIG. 2 schematically illustrates a non-limiting example of the fusion module that includes a resample, a segmentor, and a registration engine, in accordance with an aspect of an embodiment(s) herein.

FIG. 3 illustrates an example coordinate system for the ultrasound probe for resampling the v3-D US volume, in accordance with an embodiment(s) described herein.

FIG. 4 illustrates an example approach for resampling the 3-D US volume into equiangular 2-D images, in accordance with an embodiment(s) described herein.

FIG. 5A depicts a portion of 2-D slice of 3-D MR image data of a sagittal view with a contour of tissue of interest and a contour of a suspect region within the contour of tissue of interest, in accordance with an aspect of an embodiment(s) herein.

FIG. 5B depicts a portion of 2-D slice of 3-D MR image data of a transverse view with a contour of tissue of interest and a contour of a suspect region within the contour of tissue of interest, in accordance with an aspect of an embodiment(s) herein.

FIG. 6A depicts a fused image that includes an ultrasound image of a sagittal view with a contour of tissue of interest and a contour of a suspect region from the portion of 2-D slice of 3-D MR image data (FIG. 5A) registered therewith, in accordance with an aspect of an embodiment(s) herein.

FIG. 6B depicts a fused image that includes an ultrasound image of a transverse view with a contour of tissue of interest and a contour of a suspect region from the portion of 2-D slice of 3-D MR image data (FIG. 5B) registered therewith, in accordance with an aspect of an embodiment(s) herein.

FIG. 7 schematically illustrates a variation of the fusion module of FIG. 2 that further includes a primary point determiner, in accordance with an aspect of an embodiment(s) herein.

FIG. 8 depicts a fused image showing a contour of tissue of interest and a graphical indicia representing a primary target point therein, in accordance with an aspect of an embodiment(s) herein.

FIG. 9 depicts another fused image showing a contour of tissue of interest and a graphical indicia representing a primary target point therein, in accordance with an aspect of an embodiment(s) herein.

FIG. 10 schematically illustrates a variation of the fusion module of FIG. 7, in accordance with an aspect of an embodiment(s) herein.

FIG. 11 illustrates a non-limiting example of a flow chart for a computer-implemented method for generating a fused image for an ultrasound image-guided procedure, in accordance with an embodiment(s) herein.

FIG. 12 illustrates a non-limiting example of a flow chart for a computer-implemented for generating a fused image that includes a primary target point for an ultrasound image-guided procedure, in accordance with an embodiment(s) herein.

FIG. 13 illustrates a non-limiting example of a flow chart for a computer-implemented for beamforming a live transverse image from a sweep of a sagittal transducer array in the transverse direction for an ultrasound image-guided procedure, in accordance with an embodiment(s) herein.

FIG. 14 schematically illustrates a non-limiting example of a Magnetic Resonance (MR) imaging system, in accordance with an aspect of an embodiment(s) herein.

### DETAILED DESCRIPTION

Embodiments of the present disclosure will now be described, by way of example, with reference to the figures, in which a system, a method and/or instructions on a computer readable medium efficiently provide an accurate fusion of 3-D ultrasound data and pre-procedure 3-D MR image data for an ultrasound-guided procedure such as a biopsy, a therapy, and/or other procedure. In one instance, this includes acquiring the 3-D ultrasound data via a sweep of a sagittal transducer array, resampling the 3-D ultrasound data to generate a set of ultrasound image planes that are approximately orthogonal to a surface of the tissue of interest, and co-registering a contour of the tissue of interest segmented in the set of ultrasound image planes with a contour of the tissue of interest segmented in the pre-procedure 3-D MR image data to create a fused 3-D image. During the procedure, a live ultrasound image is then superimposed with contours of features segmented from a corresponding plane of the fused 3-D image.

As discussed above, an existing approach includes acquiring an ultrasound image in the sagittal plane, acquiring an ultrasound image in the transverse plane, and co-registering contours of the tissue of interest from the two orthogonal planes with a contour of the tissue of interest from a segmentation of the pre-procedure 3-D MR image data, where the two orthogonal ultrasound image planes generally are not able to show a specific point of interest simultaneously without moving the transducer to a different location where the overview of the tissue is reduced, and, as a consequence, the co-registration of the contours in the two orthogonal ultrasound image planes and the 3-D MR image data may not be accurate. The approach described herein mitigates such shortcomings and/or other shortcomings. The approach described herein further reduces the amount of memory and/or processing resources and/or processing time relative to a configuration in which the approach described herein is not utilized and the acquired 3-D ultrasound data from the sweep is processed to generate a 3-D ultrasound image that is then co-registered with the pre-procedure 3-D MR image data.

In some instances, the approach described herein further determines a primary target point in the tissue of interest based on the pre-procedure 3-D MR image data and superimposes indicia that graphically indicates the primary target point in the tissue of interest over a display of the live ultrasound image and the corresponding plane of the fused 3-D image, where the clinician can utilize the graphical indicia to advance the instrument to the primary target point for the procedure. In some instances, the approach described herein not only displays a live image in the sagittal plan, but also beamforms a live image in the transverse plane from the acquired 3-D ultrasound data that is through the primary target point and concurrently displays live images of both the sagittal and the transverse planes.

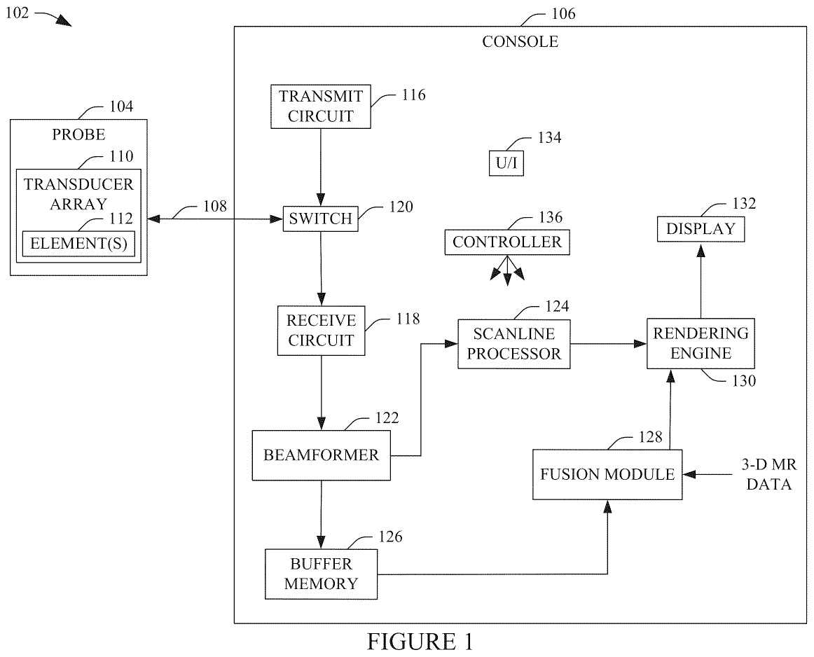

Initially referring to FIG. 1, a non-limiting example of an ultrasound system 102 is schematically illustrated. The ultrasound system 102 includes an ultrasound probe 104 and a console 106. In the illustrated embodiment, the probe 104 and the console 106 interface with each other via a communication channel 108, which includes wired (e.g., complimentary interfaces and a cable therebetween) and/or wireless technology (e.g., Wi-Fi, etc.). In another instance, the probe 104 and the console 106 are integrated in a same housing such as part of a hand-held ultrasound system, etc.

The probe 104 includes a transducer array 110. The transducer array 110 has a long axis and includes one or more transducer elements 112. Examples of suitable arrays include 64, 128, 192, 256, and/or other arrays, including larger and smaller arrays. The transducer array 110 can be linear, curved, and/or otherwise shaped, fully populated, sparse and/or a combination thereof, etc. In one instance, the transducer array 110 is configured to rotate inside of the probe 104. For instance, the transducer array 110 is spatially oriented along a sagittal plane of the probe 104 and is configured to pivot or sweep in a transverse direction over, e.g., within a range of at least 180° inside of the probe 104 by rotating the transducer array 110 in the transverse direction about an axis that is parallel to the long axis of the transducer array.

In one instance, the probe 104 may further include a transducer array rotation assembly with electro-mechanical and control components for rotating the transducer array 110 in the transverse direction inside of the probe 104 and controlling such rotation. In another instance, the transducer array 110 is not configured to rotate inside of the probe 104 and the transducer array rotation assembly is omitted, and the probe 104 is rotated manually, e.g., via a user, a robot, etc. by rotating the probe 104 in the transverse direction about an axis that is parallel to the long axis of the transducer array.

The one or more transducer elements 112 are configured to convert an excitation electrical signal to an ultrasound pressure field and convert a reflected ultrasound pressure field to an electrical signal. For example, the one or more transducer elements 112 can be selectively excited via an excitation electrical signal, which causes at least a sub-set of the transducer elements 112 to transmit an ultrasound pressure field into a scan field of view. The one or more transducer elements 112 receive echo signals and generate analog electrical signals indicative thereof. The echo signals are generated in response to the transmitted ultrasound pressure field interacting with structure, such as tissue, blood cells, etc.

The console 106 includes a transmit circuit 116 configured to generate the excitation electrical signal provided to transducer array 110 for transmitting the ultrasound pressure field. In one instance, this includes generating delays for individual elements 112 of the transducer array 110, exciting a full set of the elements 112, exciting less than the full set of the elements 112, etc. for each transmission.

The console 106 further includes receive circuit 118 configured to receive and pre-process the analog echo signals. In one instance, this includes one or more of applying a fixed amplification, applying analog time gain compensation, converting the analog echo signals to digital echo signals, down converting to shift a center frequency of the signals to baseband, decimating the signals to reduce the data rate, and/or otherwise pre-processing the signals.

The console 106 further includes a switch 120 configured to switch between the transmit circuit 116 and the receive circuit 118, e.g., by electrically connecting the transmit circuit 116 to the transducer array 110 for a transmit operation and electrically connecting the receive circuit 118 to the transducer array 110 for a receive operation. In an alternative instance, separate switches are employed such that the transmit circuit 116 has a switch and the receive circuit 118 has a different switch.

The console 106 further includes a beamformer 122. The beamformer 122 is configured to beamform, e.g., via delay-and-sum (e.g., a matched-filter beamformer, etc.) and/or other beamforming, the signals from the receive circuit 118 and construct a scanplane of scanlines of radiofrequency (RF) data or In-phase/Quadrature (IQ) data for the echoes for each receive operation.

The console 106 further includes a scanline processor 124. The scanline processor 124 is configured to perform one or more of the following: filtering (e.g., via a Finite Impulse Response (FIR) filter, an Infinite Impulse Response (IIR) filter, a Band Pass Filter (BPF), etc.), In-phase and Quadrature (I/Q) demodulation, envelope detection, dynamic range compression, compounding, dynamic range expansion, noise rejection, down-conversion, decimation, adaptive gain, etc., and output a set of scanlines as a frame/B-mode image.

The console 106 further includes a buffer memory 126. The buffer memory 126 is configured to store scanlines for volumetric or 3-D processing. For example, in one instance where the transducer array 110 is rotated (or swept) in the transverse direction to acquire 3-D volumetric ultrasound data (e.g., a set of sagittal planes angularly offset planes in the transverse direction), the scanlines corresponding to each sagittal plane are stored in the buffer memory 126 for volumetric or 3-D processing.

The console 106 further includes a fusion module 128. In one instance, the fusion module 128 is configured to co-register a contour of tissue of interest segmented from the 3-D volumetric ultrasound data in the buffer memory 126 with a contour of tissue of interest segmented in a pre-procedure 3-D MR image data. As described in greater detail below, in one instance this includes processing the 3-D volumetric ultrasound data to generate a set of planes with a certain orientation with respect to the tissue of interest and co-registering a contour of tissue of interest from the set of images with the contour of tissue of interest in the pre-procedure 3-D MR image data.

As further described in greater detail below, in some instances, the fusion module 128 is further configured to determine a primary target point in the tissue of interest based on pre-procedure 3-D MR image data, where the primary target point is visually presented via graphical indicia with the registered contours of interest, where the clinician utilizes the graphical indicia to advance the instrument to the primary target point. As further described in greater detail below, in some instances, the fusion module 128 is further configured to beamform a live transverse image from 3-D volumetric ultrasound data through the primary target point, where the beamformed live transverse image is also visually presented.

The console 106 further includes a rendering engine 130 configured to superimpose live ultrasound images over a corresponding plane of the fused image on a display 132. The console 106 further includes a user interface (U/I) 134 configured to allow a user to control an operation of the system 102. The console 106 further includes a controller 136 with a processor(s) such as a microprocessor (μP), a central processing unit (CPU), a graphics processing unit (GPU), etc., and computer readable medium, which includes non-transitory medium and excludes transitory medium (signals, carrier waves, and the like). The processor(s) is configured to execute instructions in the computer readable medium to implement one or more of the components of the console 106 and/or functions of the imaging system 102 described herein.

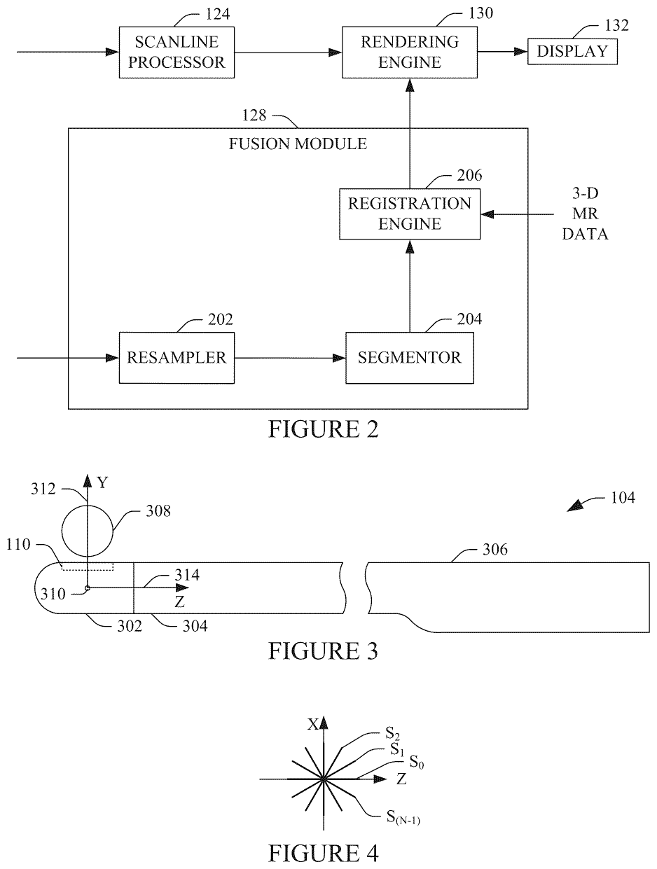

FIG. 2 schematically illustrates an example of the fusion module 128. In this example, the fusion module 128 includes a resampler 202, a segmentor 204, and a registration engine 206.

The resampler 202 retrieves and/or receives, as input, the 3-D volumetric ultrasound data in the buffer memory 126. Again, the 3-D volumetric ultrasound data includes signals for a plurality of angularly spaced sagittal planes acquire while sweeping the transducer array 110. In one instance, the 3-D volumetric ultrasound data may include signals for four hundred (400), more or less sagittal planes that are not all orthogonal to an interface of the tissue of interest. The resampler 202 is configured to resample the signals and generate a set of planes that are approximately orthogonal to the interface of the tissue of interest.

The set of planes includes at least more than two (2) sagittal planes, such as three (3) planes, nine (9) planes, twelve (12) planes, twenty (20) planes etc., but less the number of planes acquired in for the 3-D volumetric ultrasound data. For example, in one instance, the set of planes may include twelve (12) equally angularly spaced planes from hundreds of planes of the 3-D volumetric ultrasound data acquired over the sweep range such as zero (0) to one hundred and eighty (180) degrees. The resampling may include interpolation and/or other processing. This approach reduces processing resources required relative to a configuration that generates planes for the entire 3-D volumetric ultrasound data.

A non-limiting example is discussed in connection with FIGS. 3 and 4. FIG. 3 schematically illustrates a portion of an example probe and a coordinate system, and FIG. 4 schematically illustrates example resampling. Beginning with FIG. 4, in this example, the probe 104 includes a tip region 302, an elongated shaft 304 and handle 306, with the transducer array 110 in the tip region 304. In this example, the transducer array 110 is configured to rotate inside the tip region 304. A non-limiting example of such the probe 104 includes the 3D X14L Endocavity Transducer, a product of BK Medical, a company of Denmark. The user will typically have tissue of interest 308 in a center of the 3-D sweep. The coordinate (x,y,z) forms a right-hand coordinate system.

For the coordinate system, an x-axis 310 points into the page, a y-axis 312 is defined as a line in an xy-plane that splits the total sweep angle in two equal parts, and a z-axis 314 points in a direction of the shaft 304 of the probe 104. The z-axis 312 is normal to the xy-plane, the yz-plane is the sagittal body plane, and an origin is on the axis of rotation. FIG. 4 shows the 3-D US data resampled so that all N slices (S₀, S₁, S₂, . . . and S_{(N-1)}) contain the y-axis, are perpendicular to the xz-plane, and are angularly spaced with equal angular distance making the angle between the slices π/N. An example of the resampling is described in U.S. Pat. No. 10,779,798 B2 to Martins, filed on Sep. 24, 2018, and entitled “Ultrasound Three-Dimensional (3-D) segmentation,” the entirety of which is incorporated herein by reference.

In one instance, the resampler 202 is further configured to perform similar functions of the scanline processor 124, such as one or more of the following: filtering (e.g., via a FIR filter, an IIR filter, a BPF, etc.), I/Q demodulation, envelope detection, dynamic range compression, compounding, dynamic range expansion, noise rejection, down-conversion, decimation, adaptive gain, etc., and output a set of scanlines as a frame/B-mode image. Additionally, or alternatively, the resampler 202 employs the scanline processor 124 and/or other component to perform one or more of these functions.

The segmentor 204 retrieves and/or receives, as input, the set of planes generated by the resampler 202. The segmentor 204 is configured to segment a contour of the tissue of interest from the set of planes. In one instance, the output of the segmentor 204 includes a point cloud, or points in 3-D ultrasound space. Various known and/or other approaches can be used to segment the contour. For example, in one instance, the segmentor 204 includes a neural network trained to segment contours from such sets of planes. With this example, the output segmentation can be included as training data for subsequent and/or adaptive training of the segmentor 204.

The registration engine 206 retrieves and/or receives, as input, the contour segmentation of the tissue of interest from the segmentor 204 and the segmentation from the pre-procedure 3-D MR image (e.g., from T2-weighted acquisition, etc.) that includes the tissue of interest and a suspect region, such as a possible lesion(s), nodule(s), tumor(s), etc., within the tissue of interest. The MR segmentation includes a point cloud, or points in 3-D MR space. Known and/or other techniques for segmenting MR data can be utilized for the MR segmentation, including auto-segmentation techniques that follow protocols used by clinicians such as radiologists to manually segment tissue of interest and/or such regions.

The registration engine 206 is configured to co-register the segmentation of the tissue of interest from the resampled 3-D volumetric ultrasound data from the segmentor 204 and the segmentation from the pre-procedure 3-D MR image data. This includes translating the MR segmentation from MR space to ultrasound space. In one instance, this includes performing a point cloud registration for the MR coordinates into ultrasound space at least for the tissue of interest and a suspect region (i.e., possible lesion(s), nodule(s), tumor(s), etc.) segmented in the pre-procedure 3-D MR image data to generate fused 3-D data.

The rendering engine 130 retrieves and/or receives, as input, live ultrasound images form the scanline processor 124 and the fused 3-D data. The rendering engine 130 is configured to combine the live ultrasound images from the scanline processor 124 and corresponding planes of the fused 3-D data on the display 132 during the procedure.

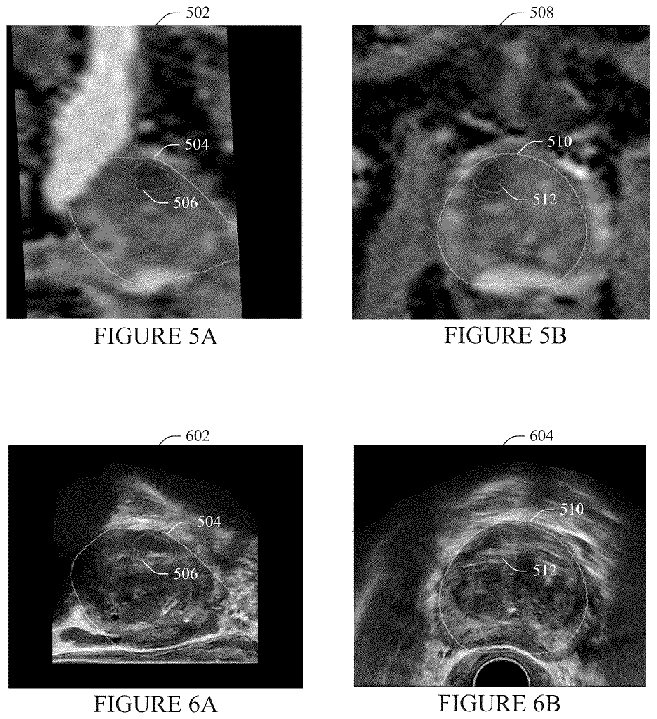

FIG. 5A depicts a portion of 2-D slice 502 of the 3-D MR image data of a sagittal view and includes a contour of tissue of interest 504 and a contour of a suspect region 506 within the contour of tissue of interest 504. FIG. 5B depicts a portion of 2-D slice 508 of the 3-D MR image data of a transverse view and includes a contour of tissue of interest 510 and a contour of a suspect region 512 within the contour of tissue of interest 510.

FIG. 6A depicts a fused image plane 602 including an ultrasound image plane of a sagittal view with the contour of tissue of interest 504 and the contour of a suspect region 506 from the portion of 2-D slice 502 shown in FIG. 5A registered therewith. FIG. 6B depicts a fused image 604 including an ultrasound image of a transverse view with the contour of tissue of interest 510 and the contour of a suspect region 512 from the portion of 2-D slice 508 shown in FIG. 5B registered therewith.

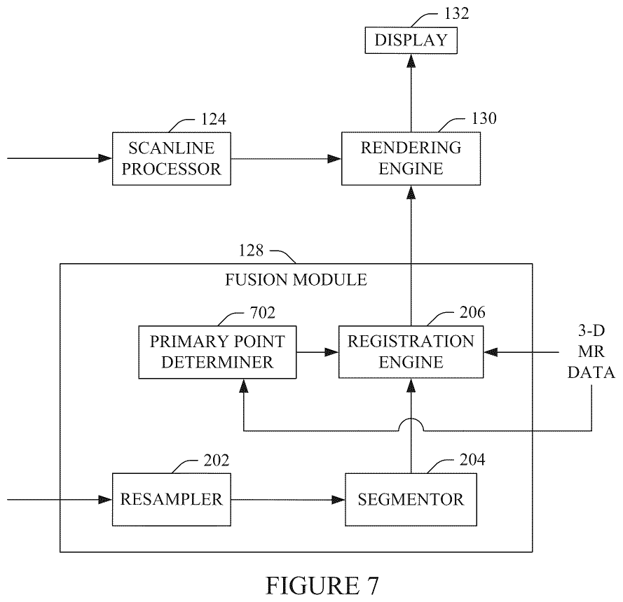

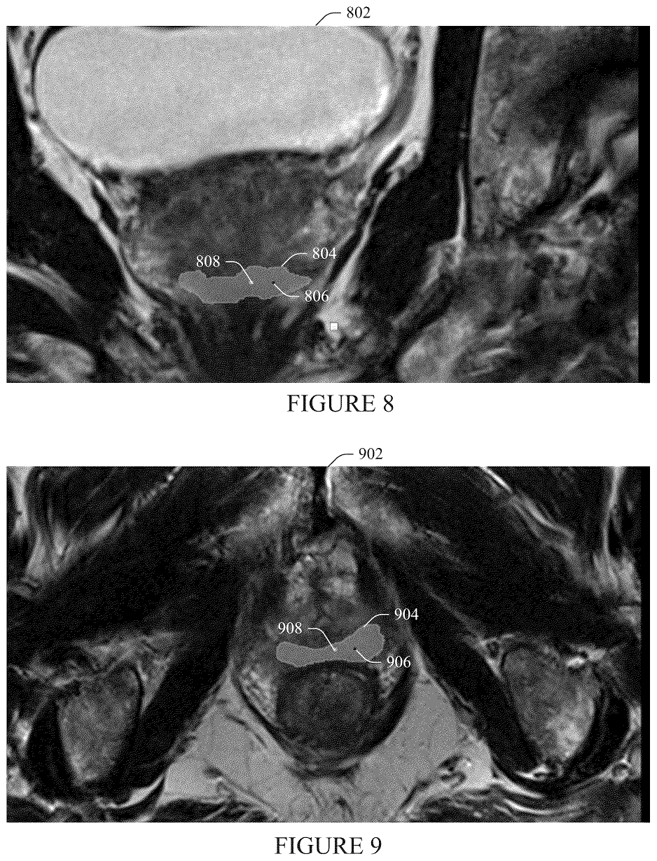

FIG. 7 schematically illustrates a variation of the example described in connection with FIG. 2. With this variation the fusion module 128 further includes a primary target point determiner 702. The primary target point determiner 702 retrieves and/or receives, as input, MR data such as a segmented T2 weighted MR image, an apparent diffusion coefficient (ADC) image, a diffusion-weighted (D-W) image, etc. The primary target point determiner 702, in one instance, is configured to determine a primary target point (based on the MR data) within the suspect region. In general, tissue of interest such a lesions, tumors, etc. are hypoechoic in ADC image and hyperechoic in D-W image.

A non-limiting example for determining the primary target point is now described. Extra-prostatic ADC-values in the ADC image are set to a high value (e.g., 2000, etc.). Extra-prostatic B-values in the D-W image are set to zero (0). A number of voxels corresponding to a predetermined volume. In one instance, the predetermined volume is based on visual inspection. In general, the size is chosen to be larger than an average lesion volume as seen by visual inspection in a number of patients. For example, for an average volume of 1.3 cm³, a suitable volume can be 1.6 cm³, 1.8 cm³, 2.1 cm³, etc. A small size allows the ADC independently to point out one or more focal lesions. For this number of voxels, voxels in the ADC image having the lowest ADC values are identified and values of the remaining voxels are set to high value (e.g., 2000, etc.). For the same voxels, voxels in the D-W image having the highest b-values are identified and values of the remaining voxels are set to zero (0).

For each voxel, a severity value is computed as a ratio of the b-value to the ADC value plus a constant. A suitable value for the constant is 5, 7, 10, 13, higher or lower. A number of voxels corresponding to another predetermined volume is determined. Likewise, the predetermined volume is based on visual inspection. In one instance, the predetermined volume is around the average size of the lesion, e.g., 1.3 cm³ in the above example. In other instances, the volume can be larger. For this number of voxels, the severity values that have the highest severity values are maintained and values of the remaining severities is set to zero (0). A connected components algorithm (e.g., one component at a time, two-pass, etc.) is then applied to create a set of unconnected lesions, nodes, tumors, etc. For each, a smoothing function (e.g., a 3-D Gaussian kernel, etc.) is applied to the severity values, and a primary target point is selected as the 3-D coordinates of a highest severity value of the smoothed severity values.

The rendering engine 130 retrieves and/or receives, as input, live ultrasound images form the scanline processor 124, the fused 3-D data, and the 3-D coordinates of the primary target point. The rendering engine 130 is configured to combine the live ultrasound images from the scanline processor 124, corresponding planes of the fused 3-D data on the display 132, and graphical indicia representing the primary target point in the contour of the suspect region on the display 132 during the procedure.

FIG. 8 depicts a fused image 802 showing a contour of a suspect region 804 and a primary target point 806. A centroid 808 of the contour of the suspect region 804 is graphically illustrated for a frame of reference. FIG. 9 depicts a fused image 902 showing a contour of a suspect region 904 and a primary target point 906. A centroid 908 of the contour of the suspect region 904 is illustrated for a frame of reference. In FIGS. 8 and 9, the indicia utilized for the primary targets points 806 and 906 is a black box for explanatory purposes. Other indicia are contemplated herein. For example, the indicia can include a shape, a pattern, a pointer, color, a user-controlled transparency level, etc.

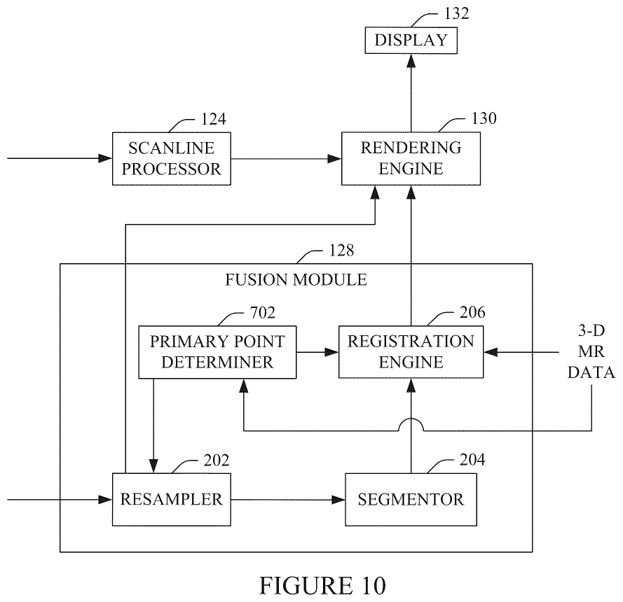

FIG. 10 schematically illustrates a variation of the example described in connection with FIG. 7. For this example, the transducer array 110 of the probe 104 is configured to not only acquire a sagittal plane during the procedure, but also perform one or more sweeps between each sagittal plane acquisition during the procedure. In this example, the 3-D volumetric data again is stored in the buffer memory 126. However, in this example, the resampler 202 further receives the 3-D coordinates for the primary target point from the primary point determiner 702.

In this example, the resampler 202 is further configured to beamform a live transverse image based on the 3-D volumetric data and the 3-D coordinates for the primary target point. The beamformed live transverse image will include a plane through the primary target point. In this example, the rendering engine 130 further receives the beamformed live transverse image. In one instance, the live sagittal image is displayed with a corresponding sagittal plane from the fused image in one viewport and, concurrently, the beamformed live transverse image is displayed with a corresponding transverse plane from the fused image in a different viewport.

In one instance, the live sagittal image can be used to track advancement of the instrument to the primary target point in the contoured suspect region, while the beamformed live transverse image can be used to visualize the instrument outside of the contoured suspect region, e.g., to align the instrument for advancement into the contoured suspect region to the primary point. In one instance, the user can control whether the beamformed live transverse image is displayed.

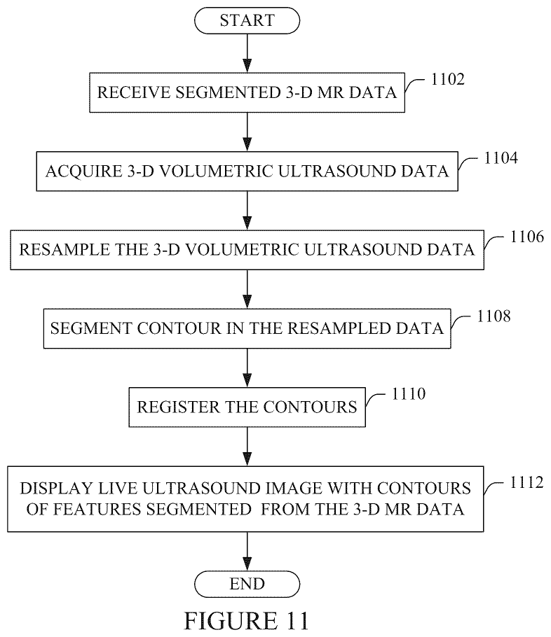

With further reference to FIGS. 1, 2, 7 and 10, the segmentor 204 can apply an auto-segmentation algorithm to segment the contour of the suspect region in each of the set of image planes, and the rendering engine 130 can display the image with the auto-segmented contours. FIG. 11 illustrates a non-limiting example of a flow chart for a computer-implemented method for generating a fused image for an ultrasound image-guided procedure. It is to be appreciated that the ordering of the acts in the method is not limiting. As such, other orderings are contemplated herein. In addition, one or more acts may be omitted, and/or one or more additional acts may be included.

At 1102, segmented pre-procedure 3-D MR image data is received, as described herein and/or otherwise. For example, in one instance 3-D MR image data from a T2-weighted, etc. acquisition is acquired. A contour of tissue of interest and a contour of a suspect region is segmented in the 3-D MR image data. In one instance, the segmentation includes a known and/or other technique, including auto-segmentation techniques that follow protocols used by clinicians such as radiologists to manually segment tissue of interest and/or such regions, and produces a point cloud, or points in 3-D MR space.

At 1104, 3-D volumetric ultrasound data of tissue of interest is acquired, as described herein and/or otherwise. For example, for the prostate, the probe 104 is placed under the prostate using live ultrasound images to guide the transducer array 110 to the prostate. The probe 104 is then activated to sweep (or rotate) the transducer array 110 in the transverse direction and acquire a plurality of sagittal planes that are angularly offset in the transverse direction, providing 3-D volumetric ultrasound data.

At 1106, the 3-D volumetric ultrasound data of tissue of interest is processed, as described herein and/or otherwise. For example, in one instance the 3-D volumetric ultrasound data of tissue of interest includes the prostate and over 100 sagittal planes. The resampler 202 resamples the signals and generates, e.g., a set of 3 to 12 equally angular spaced sagittal planes that are approximately orthogonal to a surface of the tissue of interest. As discussed herein, this approach improves registration accuracy and reduces processing requirements.

At 1108, a contour of the tissue of interest is segmented in the set of equally angular spaced sagittal planes, as described herein and/or otherwise. For example, in one instance the segmentor 204 includes and/or utilizes a trained neural network and/or other approach to segment the contour of the tissue of interest in the set of equally angular spaced sagittal planes. In one instance, the segmentation produces a point cloud, or points in 3-D ultrasound space.

At 1110, the contour of the tissue of interest segmented in the set of equally angular spaced sagittal planes and the contour of the tissue of interest segmented in the 3-D MR data are fused, as described herein and/or otherwise. For example, in one instance the registration engine 206 performs a point cloud registration that transforms the MR coordinates into ultrasound space at least for the tissue of interest and the suspect region.

At 1112, live sagittal images are superimposed with contours of features segmented from the pre-procedure 3-D MR image data, as described herein and/or otherwise. For example, in one instance the rendering engine 130 retrieves and/or receives, as input, live ultrasound images form the scanline processor 124 and the 3-D data, and combines the live ultrasound images from the scanline processor 124 with contours of features segmented from the pre-procedure 3-D MR image data on the display 132.

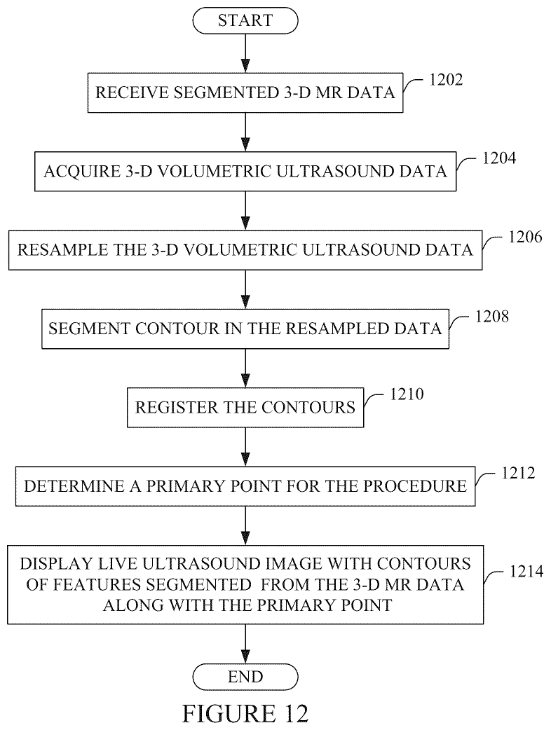

FIG. 12 illustrates another non-limiting example of a flow chart for a computer-implemented method for generating a fused image and a primary target point for an ultrasound image-guided procedure. It is to be appreciated that the ordering of the acts in the method is not limiting. As such, other orderings are contemplated herein. In addition, one or more acts may be omitted, and/or one or more additional acts may be included.

At 1202, segmented 3-D MR data is received, as described herein and/or otherwise. At 1204, 3-D volumetric ultrasound data of tissue of interest is acquired, as described herein and/or otherwise. At 1206, the 3-D volumetric ultrasound data of tissue of interest is processed, as described herein and/or otherwise. At 1208, a contour of the tissue of interest is segmented in the set of equally angular spaced sagittal planes, as described herein and/or otherwise. At 1210, the contour of the tissue of interest segmented in the set of equally angular spaced sagittal planes and the contour of the tissue of interest segmented in the 3-D MR data are fused, as described herein and/or otherwise.

At 1212, a primary target point in the suspect region is determined for the procedure, as described herein and/or otherwise. For example, in one instance the primary point determiner 702 retrieves and/or receives the segmented MR image, an ADC image, and a D-W image. The primary point determiner 702 determines a primary target point within the suspect tissue based on the MR data. For example, in one non-limiting instance, extra-prostatic ADC-values in the ADC image are set to a high value (e.g., 2000, etc.) and extra-prostatic B-values in the D-W image are set to zero (0). A number of voxels corresponding to a predetermined volume (e.g. 1.6 cm³, etc.) is determined. For this number of voxels, voxels in the ADC image having the lowest ADC values are identified and values of the remaining voxels are set to high value (e.g., 2000, etc.), and voxels in the D-W image having the highest b-values are identified and values of the remaining voxels are set to zero (0).

For each voxel, a severity value is computed based on a ratio of the b-value to the ADC plus a constant. A number of voxels corresponding to another predetermined volume (e.g. 1.3 cm³, etc.) is determined. For this number of voxels, the severity values are maintained and values of the remaining severities is set to zero (0). A connected components algorithm is then applied to create a set of unconnected lesions, tumors, etc. For each, a smoothing function (e.g., a 3-D Gaussian kernel) is applied to the severity values, and a primary target point is selected as the 3-D coordinates of a highest severity value of the smoothed severity values.

At 1214, live sagittal images are superimposed with contours of features segmented from the pre-procedure 3-D MR image data along with the primary target point, as described herein and/or otherwise. For example, in one instance the rendering engine 130 retrieves and/or receives, as input, live ultrasound images form the scanline processor 124, the contours of features segmented from the pre-procedure 3-D MR image data, and 3-D coordinates of the primary target point, and combines the live ultrasound images from the scanline processor 124 with contours of features segmented from the pre-procedure 3-D MR image data and the primary target point on the display 132.

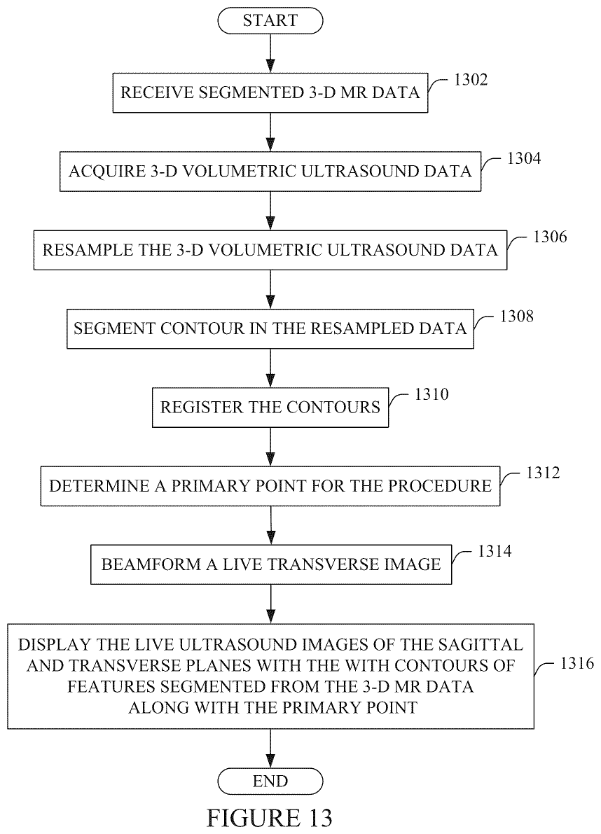

FIG. 13 illustrates another non-limiting example of a flow chart for a computer-implemented method for beamforming a live transverse image from a sweep of a sagittal transducer array in the transverse direction for an ultrasound image-guided procedure. It is to be appreciated that the ordering of the acts in the method is not limiting. As such, other orderings are contemplated herein. In addition, one or more acts may be omitted, and/or one or more additional acts may be included.

At 1302, segmented 3-D MR image data is received, as described herein and/or otherwise. At 1304, 3-D volumetric ultrasound data of tissue of interest is acquired, as described herein and/or otherwise. At 1306, the 3-D volumetric ultrasound data of tissue of interest is processed, as described herein and/or otherwise. At 1308, a contour of the tissue of interest is segmented in the set of equally angular spaced sagittal planes, as described herein and/or otherwise. At 1310, the contour of the tissue of interest segmented in the set of equally angular spaced sagittal planes and the contour of the tissue of interest segmented in the 3-D MR data are fused, as described herein and/or otherwise.

At 1312, a primary point in the suspect region is determined for the procedure, as described herein and/or otherwise. At 1314, a live transverse image is beamformed, as described herein and/or otherwise. For example, in one instance, during the procedure, the transducer array 110 is rotated to acquire 3-D volumetric data. The resampler 202 processes the 3-D volumetric data and beamforms a live transverse image based on the coordinates for the primary point. The beamformed live transverse image will include a plane through the primary point.

At 1316, live sagittal images and beamformed live transverse images are each superimposed with contours of features segmented from the pre-procedure 3-D MR image data along with and displayed, as described herein and/or otherwise. The live sagittal image can be used to track advancement of the instrument to the primary target point in the contoured suspect region, while the beamformed live transverse image can be used to visualize the instrument outside of the contoured suspect region, e.g., to align the instrument for advancement into the contoured suspect region to the primary point.

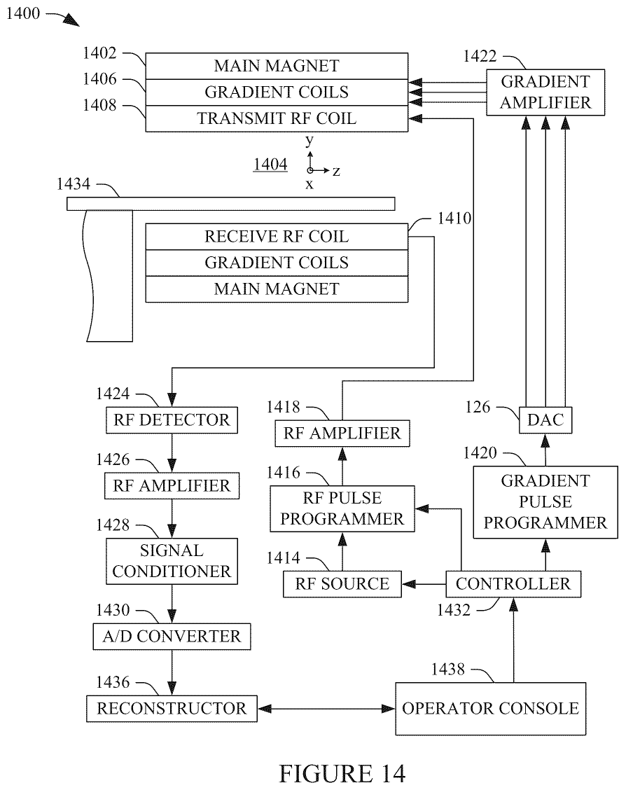

As discussed herein, MR data such as 3-D MR image data, an ADC image, and a D-W image are utilized. FIG. 14 schematically illustrates an example an imaging system 1400 configured at least for MR imaging. The imaging system 1400 includes a main magnet 1402. The main magnet 1402 is configured to provide a substantially homogeneous, temporally constant main magnetic field (B₀) in an examination region 1404. Depending on the desired main magnetic field strength and the requirements of a particular application, various magnet technologies (e.g., superconducting, resistive, or permanent magnet technologies) and/or physical magnet configurations (e.g., solenoidal or open magnet configurations) have been implemented.

The imaging system 1400 further includes gradient coils 1406. The gradient coils 1406 are configured to generate time varying magnetic gradient fields. The gradient coils 1406 include an x-gradient coil for generating a gradient field along the x-direction, a y-gradient coil for generating a gradient field along the y-direction and a z-gradient coil for generating a gradient field along the z-direction. A function of the gradient coils 1406 is to spatially encode the MR signal to differentiate signals from different locations within the body. The gradient coils 1406 are also utilized for various techniques like diffusion imaging, perfusion imaging, functional imaging, elastography imaging, angiography imaging, etc. For diffusion imaging, the gradient coils 1406 are configured to generate diffusion-sensitizing gradients that affect the image contrast.

The imaging system 1400 further includes a transmit radiofrequency (RF) coil 1408. The transmit RF coil 1408 is configured to generate RF signals that excite and/or otherwise manipulate hydrogen and/or other magnetic resonant active nuclei in an object and/or subject in the examination region 1404. The imaging system 1400 further includes a receive RF coil 1410. The receive RF coil 1410 is configured to receive magnetic resonance (MR) signals generated by the excited nuclei in the examination region 1404. The illustrated transmit RF coil 1408 and receive RF coil 1410 are volume or whole-body coils integrated in the imaging system 1400.

In another example, the RF coil 1408 is configured as the receive coil, and the RF coil 1410 is configured as the transmit coil. In another instance, the transmit RF coil 1408 and receive RF coil 1410 are part of a same transmit-receive RF coil and a switch or the like is configured to switch between transmit and receive operations. In another instance, the transmit RF coil 1408 and receive RF coil 1410 are separate from the imaging system 1400 and are installed in the imaging system 1400 for use therewith to scan the object or subject. Other coils are contemplated herein. Examples include smaller volume coils configured for extremities such as the head, etc., surface coils, etc.

The imaging system 1400 further includes an RF source 1414. The RF source 1414 is configured to generate an RF signal having a desired frequency (e.g., the Larmor frequency of the MR active nuclei under investigation). The imaging system 1400 further includes an RF pulse programmer 1416. The RF pulse programmer 1416 is configured to establish a timing and/or a shape of the RF signal generated by the RF source 1414. The imaging system 1400 further includes an RF amplifier 1418. The RF amplifier 1418 is configured to amplify the shaped RF signal to levels required by the transmit RF coil 1408 for exciting nuclei in the object or subject in the examination region 1404.

The imaging system 1400 further includes a gradient pulse programmer 1420. The gradient pulse programmer 1420 is configured to establish a timing, a strength and/or a shape of the time varying magnetic fields that are generated by the gradient coils 1406 during a scan of an object and/or subject. The imaging system 1400 further includes a gradient amplifier 1422. The gradient amplifier 1422 is configured to amplify the time varying magnetic fields to levels required by the respective gradient coils 1406. The gradient amplifier 1422 includes an independent power amplifier for each of the gradient coils 1406, including the x-gradient coil, the y-direction and the z-gradient coil. In one example, the x- and y-gradient coils respectively include a saddle (Golay) coil and the z-gradient coil includes a circular (Maxwell) coil.

A controller 1432 controls the RF source 1414, the RF pulse programmer 1416 and the gradient pulse programmer 1420. The RF pulse programmer 1416 and the gradient pulse programmer 1420 respectively control the RF amplifier 1426 and the gradient amplifier 1422 based on an imaging technique being employed for a scan of an object or subject. Examples of different imaging techniques include diffusion imaging, perfusion imaging, functional imaging, elastography imaging, angiography imaging, etc.

The imaging system 1400 further includes an RF detector 1424. The RF detector 1424 is configured to receive an analog MR signal generated by the RF receive coil 1410 during a data acquisition window having a given timing and length. The imaging system 1400 further includes an RF amplifier 1426. The RF amplifier 1426 is configured to amplify the received analog MR signal. The imaging system 1400 further includes a signal conditioner 1428. The signal conditioner 1428 is configured to condition the amplified analog MR signal, e.g., demodulate, filter, etc., the amplified MR signal. The imaging system 1400 further includes an analog-to-digital (A/D) converter 1430. The A/D converter 1430 is configured to digitize the conditioned analog MR signal, i.e., convert the conditioned analog MR signal into a digital MR signal.

The imaging system 1400 further includes a subject/object support 1434. The subject/object support 1434 includes a tabletop moveably coupled to a frame/base. In one instance, the tabletop is slidably coupled to the frame/base via a bearing or the like, and a drive system (not visible) including a controller, a motor, a lead screw, and a nut (or other drive system) translates the tabletop along the frame/base into and out of the examination region 1404. The tabletop is configured to support an object or subject in the examination region 1404 for loading, scanning, and/or unloading the subject or object. A table controller (not visible) controls the drive system.

The imaging system 1400 further includes a reconstructor 1436. The reconstructor 1436 is configured to reconstruct the digitized MR signals and generate individual axial (2-D) images and/or volumetric (3-D) image data. The MR signals include encoded imaging data (k-space), which is transformed by the image reconstruction algorithm using a Fourier transform and/or other algorithm. The 2-D images and/or the 3-D image data can be visually presented via a display monitor, filmer, etc. In one instance, the reconstructor 1436 is configured to process the MR signals and generate T2 weighted images, ADC images, D-W images, etc.

The imaging system 1400 further includes a computing system 1438. The computing system 1438 serves as an operator console of the imaging system 1400. The computing system 1438 is in communication with the reconstructor 1436. In one instance, the imaging system 1400 is configured to transfer reconstructed image to the ultrasound imaging system 102, a server, a database, a workstation, a Radiology Information System (RIS), a Hospital Information System (HIS), an Electronic Medical Record (EMR), a Picture Archiving and Communications System (PACS), etc.

The above can be implemented by way of computer readable instructions, encoded, or embedded on the computer readable storage medium, which, when executed by a computer processor, cause the processor to carry out the described acts or functions. Additionally, or alternatively, at least one of the computer readable instructions is carried out by a signal, carrier wave or other transitory medium, which is not computer readable storage medium.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include such additional elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only and are thus not intended to limit in any way the definition and/or meaning of the term “computer”. The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the invention without departing from their scope. While the dimensions and types of materials described herein are intended to define the parameters of the various embodiments of the invention, the embodiments are by no means limiting and are exemplary embodiments. Many other embodiments will be apparent to those of skill in the art upon reviewing the above description.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

Embodiments of the present disclosure shown in the drawings and described above are example embodiments only and are not intended to limit the scope of the appended claims, including any equivalents as included within the scope of the claims. Various modifications are possible and will be readily apparent to a skilled person in the art. It is intended that any combination of non-mutually exclusive features described herein are within the scope of the present disclosure. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used, as practice in some jurisdictions require them, but this should not be taken to mean that the features in the dependent claims are mutually exclusive.

What is claimed is:

1. An ultrasound imaging system, comprising:
a single transducer array having a long axis and configured to transmit and receive in either a first mode to acquire a live ultrasound image of a sagittal plane during a procedure or a second mode to acquire 3-D volumetric ultrasound data including a plurality of sagittal planes by rotating the single transducer array about an axis that is parallel to the long axis of the transducer array;
a resampler configured to resample the 3-D volumetric ultrasound data and generate a set of resampled ultrasound planes that are approximately orthogonal to a surface of tissue of interest;
a segmentor configured to segment a first contour of the tissue of interest in the set of resampled ultrasound planes;
a registration engine configured to register the first contour and a second contour of the tissue of interest in pre-procedure three-dimensional (3-D) magnetic resonance (MR) image data and create a fused 3-D image; and
a rendering engine configured to superimpose the live ultrasound image with contours of features segmented from the pre-procedure 3-D MR image data.

2. The ultrasound imaging system of claim 1, wherein the set of resampled ultrasound planes includes only three to twelve planes.

3. The ultrasound imaging system of claim 1, further including:
a primary point determiner configured to determine a primary target point for the procedure based on 3-D MR image data.

4. The ultrasound imaging system of claim 3, wherein the rendering engine is configured to superimpose indicia representing a location of the primary target point for the procedure over the fused 3-D image.

5. The ultrasound imaging system of claim 3, wherein the resampler is configured to beamform a live transverse ultrasound image through the primary target point based on additional 3-D volumetric ultrasound data obtained during the procedure.

6. The ultrasound imaging system of claim 5, wherein the rendering engine is configured to display the live ultrasound image of the sagittal plane over a sagittal view of the fused 3-D image in a first display port and display the beamformed live transverse ultrasound image over a transverse view of the fused 3-D image in a second display port.

7. The ultrasound imaging system of claim 6, wherein the rendering engine is configured to superimpose the indicia representing the location of the primary target point for the procedure over the beamform live transverse ultrasound image.

8. A computer-implemented method, comprising:
acquiring, with a single transducer array having a long axis, either a live ultrasound image of a sagittal plane during a procedure or 3-D volumetric ultrasound data while rotating the single transducer array about an axis that is parallel to the long axis of the transducer array;
resampling the 3-D volumetric ultrasound data to generate a set of resampled ultrasound planes that are approximately orthogonal to a surface of tissue of interest;
segmenting a first contour of the tissue of interest in the set of resampled ultrasound planes;
registering the first contour and a second contour of the tissue of interest in 3-D pre-procedure MR image data to create a fused 3-D image; and
superimposing the live ultrasound image with contours of features segmented from the pre-procedure 3-D MR image data on a display monitor.

9. The computer-implemented method of claim 8, wherein the set of sagittal ultrasound planes includes three to twelve sagittal planes.

10. The computer-implemented method of claim 8, further comprising:
identifying a primary point for the procedure based on MR data.

11. The computer-implemented method of claim 10, further comprising:
displaying indicia representing a location of the primary point over the fused 3-D image on the display monitor.

12. The computer-implemented method of claim 8, further comprising:
beamforming a live transverse plane through the primary point based on additional 3-D volumetric ultrasound data obtained during the procedure.

13. The computer-implemented method of claim 12, further comprising:
displaying the live ultrasound image of the sagittal plane over a sagittal view of the fused 3-D image in a first display port; and
displaying the beamformed live transverse ultrasound image over a transverse view of the fused 3-D image in a second display port.

14. The computer-implemented method of claim 12, further comprising:
acquiring a sagittal image;
acquiring a transverse image;
segmenting a first contour of the tissue of interest in the sagittal image;
segmenting a second contour of the tissue of interest in the transverse image;
displaying the first contour and the second contour;
receiving user input accepting or rejecting the sagittal image and the transverse image for a registration with the 3-D pre-procedure MR image data.

15. A computer readable medium encoded with computer executable instructions, which, when executed by a processor, cause the processor to:
acquire, with a single transducer array having a long axis, either a live ultrasound image of a sagittal plane during a procedure or 3-D volumetric ultrasound data while rotating the single transducer array about an axis that is parallel to the long axis of the transducer array;
resample the 3-D volumetric ultrasound data to generate a set of resampled ultrasound planes that are approximately orthogonal to a surface of tissue of interest;
segment a first contour of the tissue of interest in the set of sagittal ultrasound planes;
register the first contour and a second contour of the tissue of interest in 3-D pre-procedure MR data to create a fused image; and
superimpose the live ultrasound image with contours of features segmented from the pre-procedure 3-D MR image data on a display monitor.

16. The computer readable medium of claim 15, wherein the instructions further cause the processor to:
identify a primary target point for the procedure based on MR data.

17. The computer readable medium of claim 16, wherein the instructions further cause the processor to:
display indicia representing a location of the primary target point over the fused 3-D image on the display monitor.

18. The computer readable medium of claim 16, wherein the instructions further cause the processor to:
beamform a live transverse plane through the primary point based on additional 3-D volumetric ultrasound data obtained during the procedure.

19. The computer readable medium of claim 16, wherein the instructions further cause the processor to:
display the live ultrasound image of the sagittal plane over a sagittal view of the fused 3-D image in a first display port; and
display the beamformed live transverse ultrasound image over a transverse view of the fused 3-D image in a second display port.

20. The computer readable medium of claim 16, wherein the instructions further cause the processor to:
acquire a sagittal image;
acquire a transverse image;
segment a first contour of the tissue of interest in the sagittal image;
segment a second contour of the tissue of interest in the transverse image;
display the first contour and the second contour,
receive user input accepting or rejecting the sagittal image and the transverse image for a registration with the 3-D pre-procedure MR image data.
